(19) United States
(12) Patent Application Publication
(10) Pub. No.: US 2023/0331423 A1
(43) Pub. Date: Oct. 19, 2023

(54) STORAGE DEVICE AND METHOD OF STORING POWDER

(71) Applicant: FERTON HOLDING S.A., DELÉMONT (CH)

(72) Inventors: Marcel DONNET, St. Jean de Gonville (FR); Karine SAUVAGEOT-MAXIT, Corbonod (FR)

(21) Appl. No.: 18/022,777

(22) PCT Filed: Aug. 20, 2021
(86) PCT No.: PCT/EP2021/073112

§ 371 Date: Feb. 23, 2023

(30) Foreign Application Priority Data
Aug. 25, 2020 (EP) 20192676.3

(51) Int. Cl. B65D 1/02; B65D 39/08; B65D 25/40; B65B 1/04; A61K 8/19; A61K 8/26; A61K 8/34; A61Q 11/00

(52) U.S. Cl. CPC B65D 1/0207; B65D 39/08; B65D 1/0246; B65D 25/40; B65B 1/04; A61K 8/19; A61K 8/26; A61K 8/345; A61Q 11/00; A61K 2800/28

(57) ABSTRACT

A storage device for powder used for treatment of a tooth, including a housing surrounding a storage volume, and a closable opening through which the storage volume is accessible, where the storage device has a tapered shape towards the opening, where within the storage volume a powder is accommodated, and the housing is made of a rigid material.

### TECHNICAL FIELD

The present disclosure relates to a storage device for powder used for treatment of tooth and a method of storing the powder, in particular dental powder.

### BACKGROUND

Air-polishing technique is used in the dental field in order to remove stains and coloration from tooth and to remove dental biofilms. This powder blasting technique uses an abrasive powder, such as erythritol or glycine.

Using powders in the dental cavity generates dust, either during the device preparation or during the treatment. This dust is undesirable. In order to avoid this problem, soluble powders are chosen which cannot stay in the human body because they are dissolvable in water. Nevertheless, the usage of water-soluble powders induces ageing difficulties. That is, the air humidity may create a water layer on top of the particles of the powder which will then modify the particles in terms of particle size (agglomeration of particles) and flowability behavior. It can also induce powder crystallography changes which could affect powder properties. The final consequence of this humidity attack is that the powder does not work anymore in the powder chamber because of a loss of flowability properties. Further, powder clogs inside the nozzle may be generated due to particle agglomeration. In addition, abrasive or efficiency properties of the powder will be severely hampered.

Therefore, it is essential to protect the powder against humidity coming from the air. In the prior art two solutions are used. First, a hydrophobic amorphous silica is added to the powder. This is very efficient but the humidity protection is only temporary and not indefinite. Further, in case of high humid environment (60%-80% relative humidity), the humidity goes through the hydrophobic amorphous layer and starts to degrade the powder. The packaging material plays also an important role, being a physical barrier against humidity. Plastics are very advantageous because a plastic material can be deformed, and pressing such container made of plastic can help to expel the powder during a device powder chamber filling procedure. Unfortunately, every plastic is permeable to humidity leading to humidity equilibrium with the environment, with a delay of few days. The big issue with humidity is its dependency with the temperature. The higher the temperature is (30-35° C.), the more the air can afford a high humidity amount and more the ageing problem becomes important.

Second, soft pouches are used in the prior art to fill up and store the powders. These pouches have an aluminum foil sandwiched in between the plastic foils in order to ensure a better humidity barrier. However, such pouches are provided in single use dosage form. The long sealing around the pouch is the weak part where the humidity can enter and equilibrate with the external humidity. Furthermore, such kind of system only works for single use because pouches usually cannot be sufficiently closed after being opened. Further, a recommendation is provided for the user to take care of keeping the powder dry. This works well for depots, but not really for the final users, especially if they are working in highly humid regions (southeast Asia).

### BRIEF SUMMARY

Therefore, the present disclosure provides a storage device which protects a powder from aging and is useable for an end user.

According to a first aspect of the present disclosure a storage device for powder used for treatment of tooth is provided, the storage device comprising: a housing surrounding a storage volume, and a closable opening through which the storage volume is accessible, wherein the storage device has a tapered shape towards the opening, and wherein within the storage volume powder is accommodated, characterized in that the housing is made of a rigid, non-plastic material.

According to an aspect of the disclosure, the powder has hygroscopic properties such that humidity of ambient air may lead to a water film on a surface of particles of the powder. As discussed above, this deteriorates the powder (a so-called aging effect). In order to protect the powder from such aging, according to an aspect of the present disclosure, a storage volume in which the powder is accommodated is provided. The storage volume is surrounded by a housing. The housing is a closed container. Further, the housing has preferably only one opening. This provides the effect that only one opening has to be hermetically sealed so as to prevent humidity from entering into the storage volume.

In order to provide a sufficient barrier for preventing humidity from entering into the storage volume, the housing of the storage device is made of a rigid material. On the other hand, in the prior art, there is known a container made of flexible material in order to easily expel the powder out of the container by deforming the container. The housing of the present disclosure cannot be readily deformed by a user because it is made of a rigid material. Rigid material is, according to one aspect of disclosure, a metallic material having a high rigidity such that a human cannot readily deform the housing. Further, the rigid material has a low elasticity relative to plastic, that is, when a large load is applied, the housing deforms mainly plastically. According to the disclosure the rigid material comprises glass or ceramic or a mixture of the above materials.

In order to improve the dispensability of the powder, according to an aspect of the present disclosure, a tapered shape of the storage device is provided. That is, the housing has a conical shape, wherein the opening is arranged at a location of the housing having the smallest cross section. In other words, the opening is provided at a point on the housing where the housing tapers to a small cross section like a point. As a result, dispensing the powder out of the storage device may be easily performed by inclining the storage device. Further, the powder outflow is well regulable such that a user may dispense the powder out of the storage device without the need for additional tools like a spoon or a funnel. That is, due to the shape of the housing, the powder may be advantageously poured by inclining the storage device.

According to another aspect of the disclosure, the opening is closable by a lid element. The lid element is part of the storage device and may be moved from a first position in which the opening is closed by the lid element to a second position in which the opening is opened such that the storage volume is accessible. Further, the lid element is attached to the storage device by means of an integral hinge. Alternatively, the lid element is a separate element and may be completely removed from the storage device. As a result, in any case, the storage device may be hermetically closed by the lid element. Hence, the powder is well protected even if powder is discharged occasionally. According to a further aspect of the present disclosure, the lid element is pretensioned by a tensioning element that urges the lid element in the first position. The tensioning element is arranged at the opening. Further, the tensioning element is fixed to the housing. According to an alternative aspect of the disclosure, the tensioning element is removably attached to the storage device. For example, the tensioning element may be attached to the storage device by clamping. Thus, the time in which the storage device is open may be as short as possible. Therefore, the powder is well protected against humidity attacks.

According to a further aspect of the disclosure, the storage volume is sized such that a powder quantity accommodated within the storage device is in a range of 10 g to 1000 g, preferably 20 g to 500 g. As a result, the storage device can store an appropriately large amount of powder while at the same time the powder is prevented from being deteriorated. The powder has a grain size in a range of 10 μm to 70 μm.

The main idea of the present disclosure is to store powder in a rigid housing in order to protect the powder from water coming from the ambient air. Further, in order to improve the suitability for everyday use, the storage device has a specific shape and configuration such that a user may directly use the powder stored within the inventive storage device e.g. fill it into discharge device, without the need for additional tools or complicated processes.

Preferably, the rigid material is air tight and/or humidity tight.

According to an aspect of the present disclosure, air tight is a state in which no air may pass the wall of the housing. Further, humidity tight is a state in which no water and water vapor may pass the housing. In other words, the rigid material is diffusion resistant for H₂O molecules. As a result, the powder may be stored within the storage volume for a long term without being deteriorated due to water or vapor entering through the housing. According to the aspect of the present disclosure, the housing is air tight and/or humidity tight for a finite time span (e.g. for several weeks, several moth or several years).

Preferably, the housing has a wall thickness of at least 0.05 mm, preferably in a range of 0.3 mm to 1.2 mm, more preferably about 0.9 mm.

This ratio provides the optimal balance between a sufficient protection and material amount used for manufacturing. That is, a wall thickness being in the above range, provides a sufficient barrier against humidity and relatively low manufacturing costs. Further, this range is known to be sufficiently air tight and/or humidity tight.

Preferably, the storage has a cylindrical shape, wherein the opening is provided at one axial side of the cylindrical storage.

According to a further aspect of the disclosure, in a sectional view of the housing along its extension axis, the side walls are parallel to each other. As a result, the storage device is smoothly operable by a user. That is, by inclining the storage device, the powder is smoothly transferred within the storage volume to the opening. Further, the powder may be completely discharged out of the housing because there are no edges or corners which might retain any powder. Accordingly, the user can accurately and completely dispense the powder out of the storage device.

Preferably, the opening is a circular opening and the diameter of the opening is in a range of 0.01 to 0.5, preferably of 0.1 to 0.3, times the mean diameter of the cylindrical storage device.

According to a further aspect of the disclosure, the opening of the storage device has a relation to the diameter of the storage device. In said relation the optimum between dispensing ability and prevention of deterioration due to humidity is reached.

Preferably, the tapered shape is a at least a two staged tapered shape, preferably at least a two staged conical shape.

According to another aspect of the disclosure, the tapering or diminution of the cross section towards the opening is constant. Further, the tapered shape is subdivided in a first tapered portion and a second tapered portion. The first tapered portion is connected to the second tapered portion by a straight portion (i.e. the straight portion has a constant cross section). Alternatively, the first tapered portion and the second tapered portion are directly connected to each other. The first tapered portion and the second tapered portion have the same degree of tapering. That is, the cross-section reduction of the first tapered portion and of the second tapered portion is the same. Alternatively, the first tapered portion and the second tapered portion have different degrees of tapering, respectively. In particular, the second tapered portion located adjacent to the opening has a larger degree of tapering.

According to another aspect of the disclosure, the tapering is realized in a curved manner. Accordingly, the tapering is conical, for example. Accordingly, the powder is smoothly discharged out of the storage device. In more detail, using the two staged tapered shape a creation of a blockage due to powder agglomeration in a tapered section is reliably avoided. That is, a stable arc formation of the particles in the area of the tapered section is prevented. As a result, the powder flows out of the storage device in a continuous and smooth manner. According to a further aspect of the disclosure, the tapered shape is composed of a plurality of tapered portions. Accordingly, a smooth discharge of powder is guaranteed even if the grain size of the powder is relatively small.

Preferably, the rigid material is aluminum.

Therefore, according to an aspect of the disclosure, the storage device is relatively light weight. Hence, a transport and handling of the storage device is favorably easy. Further, aluminum is a good barrier for humidity. Thus, the powder is well protected within a housing made of aluminum.

According to a further aspect of the disclosure, the mean diameter of the cylindrical storage is in a range of 0.2 to 1, preferably 0.4 to 0.8, times the mean height of the cylindrical storage. Accordingly, the powder may be stored over a long time because in the above ratio the surface to volume ratio is in an optimal range such that the most volume is provided while the surface of the volume is as small as possible.

Preferably, the opening has a spout such that the powder can be dispensed out of the storage device in a defined manner.

According to a further aspect of the disclosure, the spout is an open duct. Further, the spout is an extension of an edge of the opening. The spout is arranged at the opening so as to be opposite to the tensioning element. In addition, the spout has a tapered shape towards its tip. Therefore, a user may fill the powder even into small openings using the storage device. Moreover, the spout is made of a flexible material so as to be adaptable to openings of containers to be filled with powder. Accordingly, the storing device can be used to refill a plurality of different devices. In addition, due to the open spout, the user may easily see how much powder is discharged out of the storage device and may thus adapt the inclination of the storage device accordingly. As a result, spilling the powder may be avoided.

Preferably, the storage device has a shape of a bottle, preferably a cylindrical bottle.

As a result, the storage device is especially easy operable for a user. In addition, the storage device is easy to transport and to store. Further, the storage device has a standardized shape and size such that it can be manufactured with existing manufacturing machinery and can be transported with existing transport devices.

Preferably, the opening is covered by a sealing operculum, preferably hermetically covered.

According to a further aspect of the present disclosure, the storage device is covered by the sealing operculum for transport and delivery to the end user. The user opens the sealing operculum and closes the opening by means of the lid element and optionally attaches the tensioning element to the storage device. The sealing operculum is a disposable element. That is, sealing operculum is used only once and cannot be attached to the opening again once it is removed. As a result, the lid element and the tensioning element may be reused each time a new storage device is used by a user. Further, the sealing operculum is specifically configured to withstand the loads occurring during packing and transport of the storage device. On the other hand, the lid element and the tensioning element are specifically configured to cope with the requirements occurring during use and storage of the storage device. Consequently, the storage device can protect the powder from the initial filling until the powder is used by an end user. Further, the sealing operculum is relatively cheap as compared to the lid element. As a result, costs are reduced by transporting and storing the storage device having the sealing operculum instead of the lid element. That is, the lid element has to be provided only once and may be attached to the storage device once the sealing operculum has been removed.

According to a further preferred embodiment, the sealing operculum is attached to the storage device by means of an adhesive. Therefore, the sealing operculum is optimally fitted to the storage device, in particular to the opening of the storage device. Further, the sealing operculum functions also as an indication whether the opening is properly closed and sealed by the sealing operculum or not. Therefore, an end user may easily see whether the seal is intact or not.

Preferably, the sealing opercula is made of aluminum.

Accordingly, the sealing operculum may provide the same protection against humidity as the housing which is also made of aluminum. Further, the sealing operculum made of aluminum may be favorable attached to a housing also made of aluminum.

Preferably, the opening has a thread to which a dispensing device and/or a lid element can be screwed so as to releasably close the opening.

According to an aspect of the disclosure, the dispensing device further improves the dispensability of the storage device. Preferably, both the dispensing device and the lid element uses the same thread. Therefore, the storage device is usable in various manners.

Preferably, the storage device has collar surrounding the opening and protruding from the storage device.

According to a further aspect of the present disclosure, the collar protects the surface from coming in touch with foreign elements. That is, during a transport the sealing operculum may be protected from being damaged by mechanical influences. Further, the collar is used to stack a plurality of storage device onto each other. Therefore, the bottom of the storage device is configured to fit onto the collar of another storage device. As a result, the storage device may be favorable stored and transported without the need for additional packages.

Further, the collar is also used to simplify the dispensing of the powder. That is, the collar may be used as an extension to more precisely pour out the powder off the storage device.

Preferably, the powder is a dental powder and is preferably sodium bicarbonate, glycine, calcium carbonate, aluminum trihydroxide, erythritol, hydroxylapatite, threhalose or tagatose.

According to an aspect of the disclosure, a dental powder is provided that removes stains and coloration from tooth. Further, such dental powders may also remove dental biofilms. In addition, the powder is harmless for a user and for the patient because it does not stay within the human body due to its solubleness in water. Further, the above powders are suitable to be used in basting devices that are configured to blast the powder onto a tooth to be treated. Therefore, the above powders have the suitable flow properties.

According to a further aspect of the present disclosure, a method of storing a powder used in treatment of tooth is provided, the method comprising: providing a storage device having a rigid housing surrounding a storage volume, and an opening that can be closed by a lid element and through which the storage volume is accessible, filling the powder into the storage device, and closing the storage device in a hermetically manner.

According to an aspect of the disclosure, the storage device is closed by a sealing operculum directly after the process of filling is finished. Therefore, the amount of vapor coming in contact with the powder may be reduced. Alternatively, the storage device is closed with the lid element which provides a hermetically sealed storage device, too.

Preferably, the step of filling takes place under a predetermined humidity condition.

According to a further aspect of the disclosure the filling of the storage device takes place in a controlled room in which the humidity is adjusted to be as low as possible. Thus, the air volume still existing within the storage device even if it is filled up with powder, has a relatively low humidity as compared to the normal ambient air.

According to an aspect of the present disclosure, the use of a rigid housing (e.g. a housing made of aluminum) for storing powder, in particular of dental powder, is advantageous to avoid a deterioration (i.e. aging) of the powder. Further, it is preferable that such housing is shaped so as to simplify the process of dispensing the powder off the storage device.

The advantages and features described in connection with the device are also applicable to the method, the use and vice versa.

Wherever not already described explicitly, individual embodiments or their individual aspects and features can be combined or exchanged with one another without limiting or widening the scope of the described disclosure, whenever such a combination or exchange is meaningful and in the sense of this disclosure. Advantages, which are described with respect to one aspect of the present disclosure are, wherever applicable also advantages of other aspects of the present disclosure.

### BRIEF DESCRIPTION OF THE DRAWINGS

In the drawings is:

FIG. 1 a sectional view of a storing device according to the present disclosure,

FIG. 2 perspective view of a storage device according to an embodiment of the present disclosure,

FIG. 3 a sectional view of a storing device according to the present disclosure, and

FIG. 4 a sectional view of a storing device according to the present disclosure.

### DETAILED DESCRIPTION

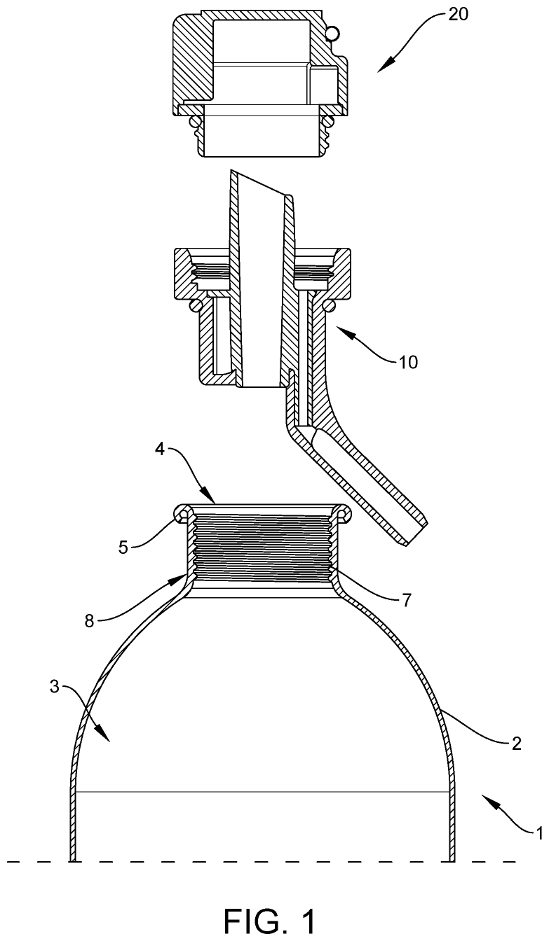

FIG. 1 is a sectional view partly showing a storage device 1. Further, in FIG. 1 there are also depicted a dispensing cap 10 and a lid element 20 detached from the storage device. In the present embodiment the storage device 1 has the shape of a bottle and is made of aluminum. The storage device 1 is formed by a housing 2 surrounding a storage volume 3. The storage volume 3 accommodates powder (not depicted in the figures). An opening 4 is provided at an axial end of the storage device 1. In other words, the opening 4 is arranged such that it forms the lowest point of the storage device 1 if the storage device 1 is turned over.

Thus, the whole powder stored within the storage device 1 may be discharged. Further, the opening 4 has a collar 8 surrounding the opening 4 in a ring like manner and protruding away from the housing 2. Within the collar 8 an internal thread 7 is formed which is configured to be engaged with a corresponding external thread of the lid element 20 or of the dispensing cap 10. The housing 2 has a wall thickness of about 0.9 mm. Thus, a humidity tight housing 4 is provided that appropriately protects the powder within the storage volume 3 from being negatively influenced by humidity coming from the ambient air. The housing 2 has a tapered shape towards the opening 4. As a result, powder stored within the housing may be easily poured out of the opening 4 by inclining the storage device 1. Therefore, according to the present embodiment it is not necessary to deform the storage device in order to pour out the whole powder.

In a further, not depicted embodiment, the storage volume 3 includes ribs within the housing 4. The ribs are configured to guide the powder within the housing 2 in case the storage device 1 is inclined. As a result, the dispensing of the powder may be performed in a well controllable manner without the occurrence of powder flushes.

Further, in FIG. 1 there is shown the dispensing cap 10. The dispensing cap 10 is used to dispense powder out of the storage device 1 in a defined and regulated manner. That is, the dispensing cap 10 includes a powder passage for discharging powder and a ventilation passage for introducing air into the storage volume 3 during a dispensing process. The powder passage has a smaller cross section as compared to the opening 4. As a result, the powder can be dispensed in a regulable manner. Further, the dispensing cap includes an external thread that fits to the thread 7 of the collar 8. Hence, the dispensing cap 10 can be removably fixed to the storage device 10.

In addition, in FIG. 1 the lid element 20 for closing the opening 4 of the storage device 1 or the opening (i.e. the passages) of the dispensing cap 10 is depicted. That is, the lid element 20 has the same external thread as the dispensing cap 10 such that it can also be attached to the thread 8 of the collar 7.

Further, both the lid element 20 and the dispensing cap include a seal element, respectively. As a result, the storage device may be closed in a hermetically manner if the dispensing cap 10 and the lid element 20 or only the lid element 20 is attached to the storage device 1.

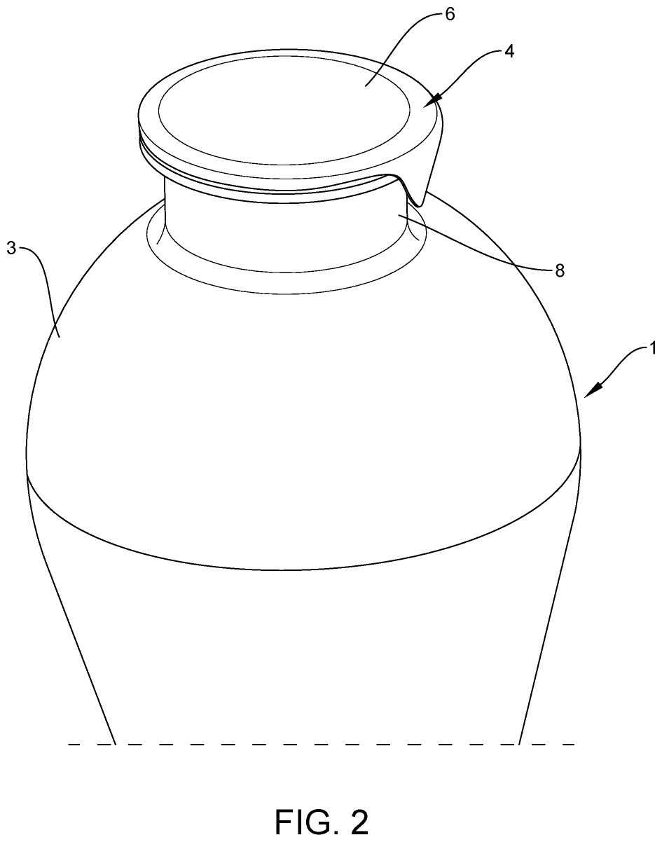

In FIG. 2 a perspective view of a further embodiment according to the present disclosure is shown. In the present embodiment the housing 2 has a different shape as compared to the previously described embodiment. That is, the housing 2 is tapered towards the opening 4 and towards a bottom of the storage device 1. In other words, the largest cross section of the storage volume is located at the half height of the storage device. In a further not depicted embodiment the largest cross section of the storage volume is located above the half height of the storage device if the storage device stands up right.

Further, in FIG. 2 the opening 4 is closed by a sealing operculum 6. In more detail, the storage device 1 depicted in FIG. 2 is sealed by means of the sealing operculum 6 directly after being filled with powder. As a result, the amount of vapor contacting the powder may be minimized. In the present embodiment, the sealing operculum 6 is attached to the opening 4 by means of an adhesive and/or application of heat. Thus, a sufficient sealing of the opening 4 is achieved. Further, sealing operculum 6 has a tear-off lip which allows an easy removement of the sealing operculum 6.

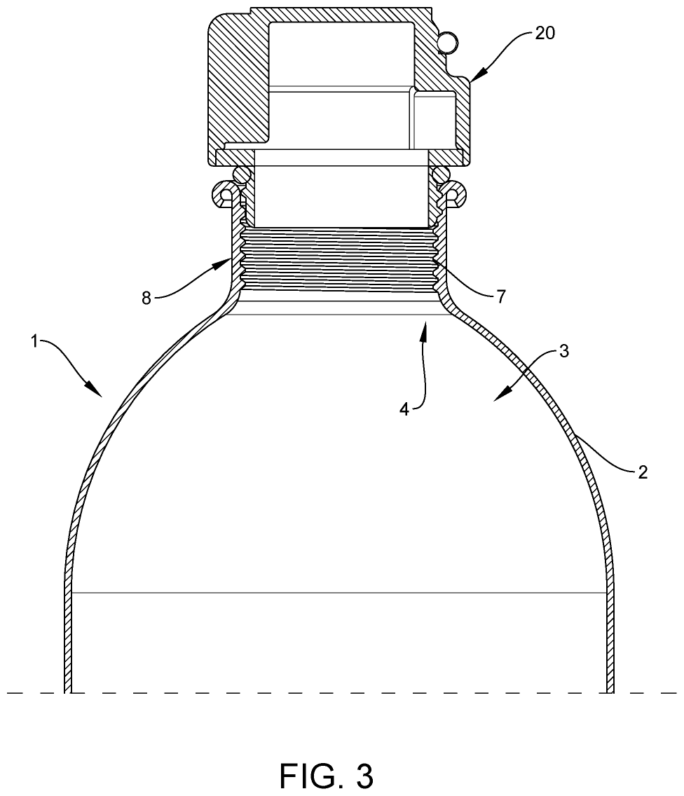

In FIG. 3 a sectional view of the storage device 1 according to a further embodiment is shown. In this embodiment, the opening 4 is directly closed by the lid element 20. The lid element 20 includes the sealing means (i.e. a sealing ring) so as to hermetically seal the storage device 1. As a result, the powder stored within the storage volume 3 may be protected from humidity coming from the ambient air. Nevertheless, the storage device 1 may be occasionally used to dispense powder out of the storage volume. After the dispensing, the opening is closed again by the lid element 20 and the powder may be further stored without being deteriorated.

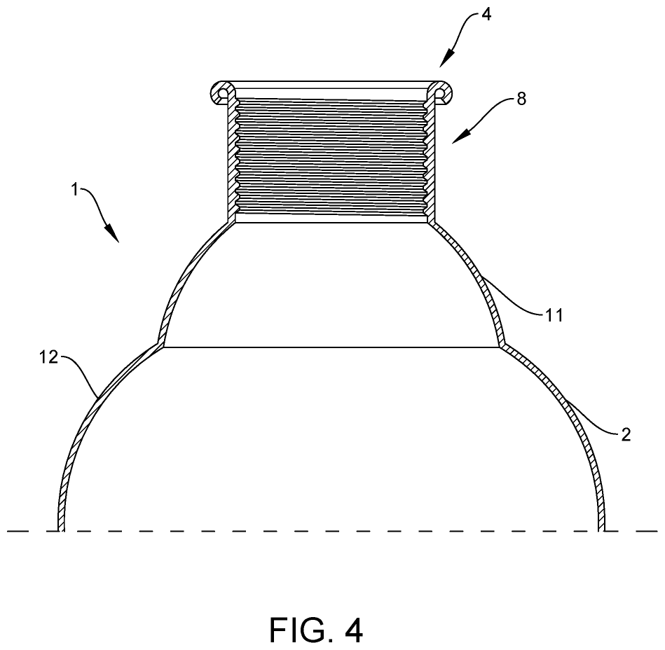

In FIG. 4 a sectional view of the storage device 1 according to a further embodiment is shown. The present embodiment differs from the above described embodiments in that the tapered shape towards the opening 4 is compounded of a first tapered portion 11 and a second tapered portion 12. The first tapered portion 11 is located adjacent to the opening 4.

The second tapered portion 12 is arranged directly adjacent to the first tapered portion 12. Both tapered portions 11, 12 have a curved shape in the cross section depicted in FIG. 4. As a result, between the first tapered portion 11 and the second tapered portion 12 an edge is located. In the present embodiment the curvature radius of the first tapered portion 11 and the second tapered portion 12 is the same. In a further not depicted embodiment the curvature radius of the first tapered portion 11 and the second tapered portion 12 differs. In this case, the curvature radius of the first tapered portion 11 is bigger as compared to that of the second tapered portion 12. As a result, the powder is discharged out of the storage device in a smooth and uniform manner.

1. Storage device for powder used for treatment of a tooth, the storage device comprising:
a housing surrounding a storage volume, and
a closable opening through which the storage volume is accessible,
wherein the storage device has a tapered shape towards the opening,
wherein within the storage volume powder is accommodated, and
wherein the housing is made of a rigid non-plastic material.

2. Storage device according to claim 1, wherein the rigid material is air tight and/or humidity tight.

3. Storage device according to claim 1, wherein the housing has a wall thickness of at least 0.05 mm.

4. Storage device according to claim 1,
wherein the storage device has a cylindrical shape, wherein the opening is provided at one axial side of the cylindrical storage device, and
wherein the opening is a circular opening and the diameter of the opening is in a range of 0.01 to 0.5 times the mean diameter of the cylindrical storage device.

5. Storage device according to claim 1, wherein the tapered shape is a at least a two staged tapered shape.

6. Storage device according to claim 1, wherein the rigid material is aluminum.

7. Storage device according to claim 1, wherein the opening has a spout such that the powder can be dispensed out of the storage device in a defined manner.

8. Storage device according to claim 1, wherein the storage device has a shape of a bottle.

9. Storage device according to claim 1, wherein the opening is covered by a sealing operculum.

10. Storage device according to claim 9, wherein the sealing opercula is made of aluminum.

11. Storage device according to claim 1, wherein the opening has a thread to which a dispensing device and/or a lid element can be screwed so as to releasably close the opening.

12. Storage device according to claim 1, wherein the storage device has collar surrounding the opening and protruding from the storage device.

13. Storage device according to claim 1, wherein the powder is a dental powder comprising sodium bicarbonate, glycine, calcium carbonate, aluminum trihydroxide, erythritol, hydroxylapatite, threhalose. or tagatose

14. Method of storing a powder used in treatment of tooth comprising:
providing a storage device according to claim 1, having a rigid, non-plastic housing surrounding a storage volume, and an opening through which the storage volume is accessible and that can be closed by a lid element,
filling the powder into the storage device, and
closing the storage device in a hermetically manner.

15. The method according to claim 14, wherein the step of filling takes place under a predetermined humidity condition.
